(19) United States
(12) Patent Application Publication
(10) Pub. No.: US 2024/0269166 A1
(43) Pub. Date: Aug. 15, 2024

(54) PAIN RELIEF COMPOSITION

(71) Applicant: TO BETTER DAYS LIMITED, London, Greater London (GB)

(72) Inventors: Adrian HOPE, Kingswear, Devon (GB); Jan LYFTOGT, Hanmer Springs (NZ)

(21) Appl. No.: 18/565,037

(22) PCT Filed: Jun. 1, 2022
(86) PCT No.: PCT/GB2022/051389

§ 371 Date: Nov. 28, 2023

(30) Foreign Application Priority Data
Jun. 3, 2021 (GB) 2107969.4

(51) Int. Cl. A61K 33/06; A61K 9/00; A61K 9/06; A61K 31/165; A61K 36/324; A61K 36/61; A61K 36/67; A61K 36/9066; A61K 47/10; A61K 47/14; A61K 47/22; A61P 29/00

(52) U.S. Cl. CPC A61K 33/06; A61K 9/0014; A61K 9/06; A61K 31/165; A61K 36/324; A61K 36/61; A61K 36/67; A61K 36/9066; A61K 47/10; A61K 47/14; A61K 47/22; A61P 29/00

(57) ABSTRACT

A formulation for treating pain is provided. In particular, the formulation includes an effective amount of Magnesium L-lactate for topical application.

The present invention relates generally to a formulation for inhibiting pain. More specifically, the invention relates to a composition comprising an effective amount of magnesium L-lactate for use in the treatment of pain.

### BACKGROUND OF THE INVENTION

According to the Sherrington definition, pain is the physical adjunct of an imperative protective reflex. In this definition, the concept of pain is termed as a protective mechanism which associates the consciousness of experiencing pain to a harmful stimulus. There are various types of nerve fibres in the body whose free endings form nociceptors which become activated in response to such harmful stimuli. The nerve fibres carry information away from the nociceptors to the central nervous system. These nerve fibres connect peripheral organs to the spinal cord but differ from each other mainly in diameter and in the surrounding myelin sheath thickness. Both these parameters control the speed at which the nerve fibres conduct nerve impulses.

Broadly, there are two main types of nerve fibres associated with pain—A-delta fibres which are myelinated resulting in faster transmission of nerve impulse causing “fast pain”, and C fibres which are non-myelinated causing “slow pain” due to slower transmission of stimulation.

Persistent pain is caused by C fibres and according to Sir Charles Sherrington—Brain, Volume 130, Issue 4, April 2007, Pages 887-894, these small ‘naked’ nerves (C fibres) can respond with a “vital protective response”, which is associated with a conscious ‘pain’ perception known as chronic or persistent pain.

Ischaemia, following an injury is the most common cause of C-fibre activation. It results in hypoxia and glycopenia/hypoglycaemia. When C-fibres run out of energy, they depolarise and trigger C-fibre spike formation, which is consciously experienced as pain. C-fibre firing causes voltage gating of TRPV1 (transient receptor potential vanilloid 1) and TRPA1 (transient receptor potential ankyrin 1) ion channels. This is followed by the release of CGRP (calcitonin gene-related peptide) and SP (substance P), two neuropeptides with extremely potent vasodilatory actions. The result is a massive increase in local blood flow at sites different from the original stimulus. It is called neurogenic inflammation which has been linked to pain and migraine.

In human skin, it is estimated that there are 100-200 C fibres per cm² which amounts to around 2-4 million in total. They are also extensively expressed in fascia surrounding muscle, bone and cartilage.

Sherrington identified two coordinating processes in the nervous system which are required to maintain a constant internal environment vital for maintaining normal nerve function. These are called activation and inhibition. Activation occurs due to depolarisation which spreads along the nerve fibre. Inhibition occurs due to repolarisation having a purely local effect which does not spread.

Activation occurs most commonly in response to ischaemia (lack of blood). From Mclver and Tanelian's studies (Anesthesiology, Volume 76, April 1992, Pages 617-623) on C fibres it is known that ischaemia causes a lack of oxygen and glucose in the neuronal environment. The nervous system comprises two group of cells, glial cells and neurons (nerve fibres). Neurons are responsible for sensing change and communicating with other neurons. Glial cells, which are not excitable, are responsible for supporting the viability of the nerve fibres, i.e., their function is to support, nourish, insulate neurons and remove waste products.

There is a metabolic division between the glial cells and nerve fibres. Metabolically, glial cells depend on a glycolytic process. Glycolysis is the first step in the breakdown of glucose to extract energy for cellular metabolism. The nerve fibres depend on an oxidative process that takes place in mitochondria for extracting even more energy in continuation from glycolysis in cytosol.

It is the lack of glucose (glycopenia/hypoglycaemia) that results in C fibres running out of energy due to the disruption of the oxidative process, unable to maintain a membrane potential of −70 mV resulting in depolarisation. This is the physiology underlying activation. It follows that correcting ischaemic perineural glycopenia will result in repolarisation. This is associated with pain relief. This process is called inhibition.

Ischaemia has an effect on both glial cells and neurons. Glial cells require glucose and neurons require pyruvate/lactate for maintaining their energy metabolism. Therefore, both these mechanisms need recovery in order to achieve inhibition.

Chronic pain is associated with conditions such as back injury, migraine headaches, arthritis, herpes zoster, diabetic neuropathy, temporomandibular joint syndrome, and cancer. Many of the currently available pain therapies are either inadequate or cause uncomfortable to deleterious side effects. Chronic pain results not just from the physical injury but also from a combination of physical, emotional, psychological, and social abnormalities. Because pain often persists in individuals even after an injury is healed, it is the ongoing pain rather than the injury that underlies the patient's disability. Untreated pain may become self-perpetuating because pain has immunosuppressive effects that leave patients susceptible to subsequent diseases.

There are many medicinal formulations available for the treatment of pain, including chronic pain. However, many analgesics currently available either target the neurotransmitters that are released at the synapses of the C fibres for pain relief or are nonsteroidal anti-inflammatory drugs which work mainly to just lower inflammation of painful areas. These drugs include expensive ingredients, require regular and frequent application, often at high doses causing side-effects, and provide relief of pain for only short periods. Many products that provide pain relief do not improve recovery from injury. Thus, the current mechanisms of pain suppression are ineffective in providing long-term relief.

Therefore, there is a need for a pain relief composition that is more effective, long-lasting, and that enhances recovery from injury even without the need of daily use. This invention relates to a pain relief composition that targets the disruption of glycolysis and oxidative process which is the root cause of activation and hence pain, as mentioned above.

### SUMMARY OF THE INVENTION

According to a first aspect, the invention provides magnesium L-lactate or a solvate thereof for use in the treatment of pain.

According to a second aspect of the invention, there is provided a pain relief composition comprising an effective amount of magnesium L-lactate, or a solvate thereof.

### DETAILED DESCRIPTION OF THE INVENTION

As used herein, the term “pain” refers to both chronic and acute pain, as well as neuropathic pain and osteoarthritic pain, and also that associated with recovery from injury, surgery, or trauma.

As used herein, the term “acute pain” refers to pain characterized by a short duration, e.g., three to six months. Acute pain is typically associated with tissue damage, and manifests in ways that can be easily described and observed. It can, for example, cause sweating or increased heart rate. Acute pain can also increase over time, and/or occur intermittently.

As used herein, the term “chronic pain” refers to pain which persists beyond the usual recovery period for an injury or illness. In one embodiment, chronic pain is the pain that lasts longer than three months. Chronic pain can be constant or intermittent. Common causes of chronic pain include, but are not limited to, arthritis, cancer, Reflex Sympathetic Dystrophy Syndrome (RSDS), repetitive stress injuries, shingles, headaches, fibromyalgia, and diabetic neuropathy.

As used herein, the term “neuropathic pain” refers to a complex, chronic pain that usually is accompanied by tissue injury and results from lesions or diseases affecting the somatosensory system. With neuropathic pain, the nerve fibres themselves may be damaged, dysfunctional or injured. These damaged nerve fibres send incorrect signals to other pain centres. The impact of nerve fibre injury includes a change in nerve function both at the site of injury and areas around the injury.

As used herein, the term “osteoarthritic pain” refers to pain resulting from osteoarthritis, a degenerative joint disease and the most common type of arthritis. It is associated with the degradation and loss of a cartilage that covers and cushions the ends of bones in normal joints. Osteoarthritis causes the cartilage in a joint to become stiff and lose its elasticity, making it more susceptible to damage. Over time, the cartilage may wear away in some areas, greatly decreasing its ability to act as a shock absorber. As the cartilage wears away, tendons and ligaments stretch, causing pain. If the condition worsens, the bones could rub against each other, causing even more pain and loss of movement.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the degree of protection desired, the formulation of the composition, the assessment of the medical situation, and other relevant factors. It is expected that the amount will fall in a relatively broad range that can be determined through routine trials.

“Topical administration” as used herein refers to application of magnesium L-lactate (or a suitable formulation thereof) to an exterior part of the body, preferably the skin.

Without being bound by any such theory, it is accepted that L-lactate enters the Citric Acid Cycle for oxidative metabolism producing large amounts of energy (34 Mol of ATP) restoring repolarisation, inhibiting pain and allowing for natural tissue maintenance, repair and renewal. Magnesium is an important co-enzyme in glycolysis and provides stability of calcium ion channels. This causes repolarisation and inhibition of pain.

### Compositions

In one aspect, the present invention is based on pharmaceutical compositions containing magnesium L-lactate that provide improved bioavailability following administration in a cream or ointment formulations. The pharmaceutical compositions of the present invention include magnesium L-lactate. This compound is suitable for administration by any route but is particularly suited for topical administration.

Magnesium L-lactate (IUPAC name—magnesium; (2S)-2-hydroxypropanoate; also known as L-Lactic acid magnesium salt, magnesium di-L-lactate) is a chemical compound having the formula

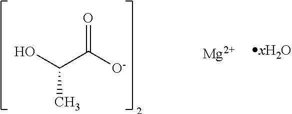

wherein x may be 0, 1 or 2. Although all forms are contemplated by the present invention, the dihydrate (x=2) is preferred.

Preferably, the magnesium L-lactate is formulated together with a carrier. The carrier may be any gel, ointment, lotion, emulsion, cream, foam, mousse, liquid, spray, suspension, dispersion or aerosol which is capable of delivering magnesium L-lactate from the formulation to the tissue. A suitable emulsifying agent may be included. A penetration enhancer may be added to enable the active agents to cross the barrier of the stratum corneum.

Preferably, the composition of the present invention includes from 1.0 to 50.0 weight percent magnesium L-lactate. More preferably, the amount of magnesium L-lactate in the composition may be from 5 to 30% w/w, preferably from 10 to 25% w/w, more preferably from 15 to 20% w/w. In one embodiment, the amount of magnesium L-lactate in the composition is 7% w/w. In another embodiment, the amount of magnesium L-lactate in the composition is 17% w/w.

### Topical Creams

In a preferred embodiment, the composition of the invention is a topical cream. Cream vehicles may include one or more components selected from the group comprising emulsifiers, emollients, preservatives and penetration enhancers. Further active ingredients may also be present.

### Saturated Fatty Acids

Saturated fatty acids selected from the group consisting of caprylic acid, capric acid, lauric acid, myristic acid, palmitic acid, stearic acid, arachidic acid, behenic acid, lignoceric acid, and cerotic acid. The preferred compositions of this invention include stearic acid, a saturated fatty acid with an 18 carbon chain and has the IUPAC name octadecanoic acid. The preferred concentration is from about 0.1 to 1.0% by weight, with a highly preferred concentration of 0.4% by weight.

### Cationic Conditioners

Cationic conditioners and emulsifiers, which may be used according to the invention, include behentrimonium methosulfate, cetrimonium chloride, myristamidopropyl PG-dimonium chloride phosphate, brassicyl isoleucinate esylate.

The preferred composition of this invention includes behentrimmium methosulfate, with quantities of cetyl alcohol and butylene glycol, i.e., Incroquat™ Behenyl TMS-50 (Croda International, Plc). It assists in forming cationic skin care emulsions that impart wash resistant moisturizing benefits and a soft, smooth skin feel. Broadly, the concentration is from about 0.1 to 10.0% by weight, with a preferred concentration of 1.0% to 10% by weight, with a highly preferred composition at about 4% by weight.

Other cationic conditioners that can be used are Incroquat behenyl TMS, Incroquat behenyl TMS 50, cetrimonium chloride, distearyldimonium chloride, and stearalkonium chloride.

### Emollients

Emollients selected from the group consisting of silicone fluids, emollient esters, emollient ethers, natural (avocado, coconut, safflower, etc.) and synthetic oils (mineral). The preferred compositions of this invention include dimethicones, preferably linear, polydimethylsiloxanes with viscosities ranging from 1.5 cSt to 20 million cSt. They are clear, colorless, odorless and inert fluids and are characterized by their softening effect on skin and hair. They are hydrophobic without restricting respiration of the skin that makes them beneficial in skin creams and lotions. Broadly, the concentration is from about 0.1 to 10.0% by weight, with a highly preferred composition at about 0.15% by weight.

### Fatty Alcohols

Preferred fatty alcohols are selected from the group consisting of tert-Butyl alcohol, tert-Amyl alcohol, 3-Methyl-3-pentanol, ethchlorvynol, capryl alcohol, 2-ethyl hexanol, pelargonic alcohol, capric alcohol, undecyl alcohol, lauryl alcohol, tridecyl alcohol, myristyl alcohol, pentadecyl alcohol, cetyl alcohol, palmitoleyl alcohol, heptadecyl alcohol, stearyl alcohol, nonadecyl alcohol, arachidyl alcohol, heneicosyl alcohol, behenyl alcohol, erucyl alcohol, lignoceryl alcohol, ceryl alcohol, 1-heptacosanol, montanyl alcohol, cluytyl alcohol, myricyl alcohol, melissyl alcohol, geddyl alcohol, and cetearyl alcohol, oleyl Alcohol and octyldodecanol.

The preferred compositions of this invention include 2-octyldodecanol, Eutanol® G (Cognis). This is a “medium spreading” emollient that is hydrolysis stable and typically used in formulations where a wide pH range is needed e.g. deodorants, antiperspirants, cosmetic and pharmaceutical skin care preparations and for hair remover formulations.

The preferred compositions of this invention also include cetyl alcohol, also known as 1-hexadecanol and palmityl alcohol, is a fatty alcohol with the formula CH₃(CH₂)₁₅OH.

Broadly, the concentration of fatty alcohol is from about 0.1 to 3.0% by weight, with a preferred composition containing 0.1 to 3.0% by weight 2-octyldodecanol and 0.1% to 2.0% phenoxyethanol, with the highly preferred composition containing 3.0% by weight 2-octyldodecanol and 0.5% phenoxyethanol.

### Cooling Agents

The compositions of this invention further include a cooling agent, which provides, when applied to the skin, a cooling or soothing feeling. The highly preferred cooling agent used in this invention is menthyl lactate. Other cooling agents, which may be used according to the invention, include, but are not limited to natural and synthetic menthol derivatives. Menthyl lactate (Frescolate®) has a mild, cooling, fresh, minty, somewhat burnt sugar like and sweet menthol taste profile. It is used in minty compounds for various purposes such as toothpaste, chewing gum and tobacco. It is also used in confections, beverages, and as a peppermint booster in oral care products. Menthyl lactate is produced in two forms, a white crystalline powder and fused material; both forms are used in various applications. It has been discovered that in a cationic carrier the antimicrobial agent is potentiated and synergized with menthyl lactate cooling agent. Broadly, the concentration is from about 1.0 to 10.0% by weight, with a highly preferred composition at about 4.5% by weight.

In an embodiment, the composition may further comprise an effective amount of vitamin D. The definition of “effective amount” is same as above. Vitamin D helps in repolarisation of the C fibres and hence, it aids in stimulating the inhibition more effectively. In one embodiment, vitamin D may be used in the form of a patch.

Preferably, the composition of the present invention may include up to about 40,000 International Units per ml of vitamin D. More preferably, the amount of vitamin D in the composition may be up to about 30,000 International Units per ml.

In another embodiment, the composition may include various natural components that alleviate, reduce, and/or eliminate pain in a safe manner. Examples of such ingredients are aloevera, boswellin, black pepper, turmeric, capsicum, wintergreen oil, camphor oil, menthol oil, onion, lemon, alfalfa, apple cider vinegar, apple seed oil, apricot oil, B-5, B-6, B-12, beta carotene, black seed oil, borage seed oil, bromelain, chanca Piedra, chasterberry extract, collagen, Du Zhong, echinachea, eucalyptus, fever few, GABA, GSE, hemp oil, CBD, L-Arginine, lavender, L-Lysine, magnesium, MSM, omega-3, peppermint oil, red clover tea, rosemary oil, sea buckthorn oil, curcumin, tea tree oil, witch hazel, yohimbe, garlic, any other essential oil, or combinations thereof.

The compositions of the present invention preferably include from 5 to 25 weight percent water-insoluble carrier selected from the group consisting of the following: hydrophobic petroleum distillates such as mineral oil, petrolatum, or ceresin; silicone derivatives such as cyclomethicone and dimethicone; emollient alkyl or aryl esters of fatty alcohols; vegetable oils and their hydrogenated derivatives; or mixtures thereof. The compositions of the present invention may further include about 1 to about 5 weight percent water-soluble carrier selected from the group consisting of glycerin, propylene glycol, other polyhydric alcohols, or mixtures thereof.

In an embodiment, the composition may include surfactants that are effective for aiding in emulsification. Preferably, the surfactant is selected from group comprising alkyl-modified ether adducts of dimethicone, hydrophilically modified alkyl fatty acid esters of polyols, or mixtures thereof. Preferred emulsifiers may be selected from caprylic triglycerides, capric triglycerides (edible), cetearyl alcohol, glyceryl monostearate-non gmo palm oil-powder, ceteareth-20, cetyl alcohol, or mixtures thereof.

The composition may further include antimicrobials and/or preservatives. Compositions used on the skin should be protected against the growth of potentially harmful microorganisms. Any preservative for topical cosmetic or pharmaceutical compositions known in the art may be used with the composition of the present invention. Antimicrobials useful in the invention may include parabens (such as methylparaben, ethylparaben, propylparaben, or butylparaben), benzyl alcohol, tetrasodium EDTA (ethylenediaminetetraacetic acid), or any other antimicrobial known in the art. A particularly preferred preservative may be selected from 2 phenoxyethanol3(2-ethylhexyloxy)propane-1,2-diol, imidazolidinyl urea, or a combination thereof.

Additional ingredients, such as fragrance, colorants, tocopherol acetate (Vitamin E), emollients, moisturizing agents, or the like may optionally be included in the composition of the present invention. The composition may include about 0.01 to about 2.0 weight percent of these optional ingredients. These optional ingredients should be non-irritating to the skin.

In an embodiment of the invention, the composition is intended for use in the management and treatment of chronic pain and pain relief of joints, nerves, bones and muscle areas, including surgical scars.

In a preferred embodiment, the composition may be a topical analgesic that is easy to apply providing convenient therapy for muscle aches, arthritis pain, and joint stiffness. The composition may be provided in a variety of formulations and viscosities, such as a lotion, foam, paste, body rub, cream, gel, serum, ointment, liniment, stick-type solid, sprayable solution, topical dermal patch, or the like for convenient application by the user. Additionally, it is contemplated that depending on the form of the composition, it may be rubbed, poured, or sprayed onto the skin, and it may be applied to the user's skin by hand, using an applicator, or by spray. Such a composition is readily absorbed by the skin and can be applied to the skin on an as needed basis for the desired pain relieving effect.

In a more preferred embodiment, the composition may be provided in the form of a cream which is applied directly to the problem area and when used consistently and regularly, provides a pain inhibiting effect which is longer than the length of the applications. The potential areas of application include surgical scaring and well as long term chronic conditions.

It is further contemplated that the composition of the present invention may be provided in combination with a variety of other skin treatment compositions, such as medicated lotions, suntan lotions, moisturizers, anti-aging compositions, warming cream, cooling cream, the like, or mixtures thereof.

In some embodiments, the formulation may be applied as part of or impregnated within a dressing that is applied to an injury, such as a bandage, poultice, cold or hot compress or a plaster cast.

In a preferred embodiment of the invention, the composition may be provided in a variety of formulations with the following viscosities: about 20 to about 4000 cps for sprayable liquids, serums, and the like; about 4000 to about 20,000 cps for lotions, liniments, and the like; about 20,000 to about 300,000 cps for creams, gels, pastes, ointments, and the like; and sedentary for stick solids.

In another embodiment, the composition may be provided for use in the treatment of ischaemia, Diabetic neuralgia, fibromyalgia, Chronic Fatigue Syndrome, Complex Regional Pain Syndrome, and Covid-19 related ‘long haul’ symptoms as these are conditions caused by skin C fibres.

Preferred embodiments of the invention will now be described by way of example only.

### Example 1

In a first example, the invention relates to a cooling cream formulation comprising 17 weight % of magnesium L-lactate, 10 weight % of aloe vera, 1 weight % of boswellin, and 0.5 weight % of eucalyptus essential oil. The rest of the composition is made up of the bulking agents, emollients, emulsifiers, and preservatives.

SPECIFICATION - Formulation 1 Cooling Cream
Product Name: MAGNESIUM LACTATE CREAM (PARABEN FREE)
Batch Size 1 Kg

| Ingredients in W.D Order | %/Mix | Function |
|---|---|---|
| DE IONISED WATER | NA | Bulking Agent |
| MAGNESIUM LACTATE | 17.000 | Active |
| CAPRYLIC.CAPRIC/ | NA | Emulsifier |
| TRIGLYCERIDES (EDIBLE) | | |
| ALOE VERA | 10.000 | Active |
| CETEARYL ALCOHOL | NA | Emulsifier |
| GLYCERYL MONOSTEARATE- | NA | Emulsifier |
| NON GMO PALM OIL-POWDER | | |
| ISOPROPYL MYRISTATE (IPM) | NA | Emollient |
| CETEARETH-20 | NA | Emulsifier |
| CETYL ALCOHOL | NA | Emulsifier |
| 2 PHENOXYETHANOL3(2- | NA | Preservative |
| ETHYLHEXYLOXY)PROPANE- | | |
| 1,2-DIOL | | |
| BOSWELLIN | 1.000 | Active |
| EUCALYPTUS ESSENTIAL OIL | 0.500 | Active |
| IMIDAZOLIDINYL UREA | NA | Preservative |

Characteristics
Colour: YELLOW CREAM
Odour: CHARACTERISTIC
Shelf Life: 2 Years Minimum

### Example 2

In a second example, the invention relates to a warming cream formulation comprising 17 weight % of magnesium L-lactate, 0.5 weight % of black pepper, 0.5 weight % of turmeric, and 0.01 weight % of capsicum. The rest of the composition is made up of the bulking agents, emollients, emulsifiers, and preservatives.

SPECIFICATION - Formulation 2 Warming Cream
Product Name: MAGNESIUM LACTATE CREAM (PARABEN FREE)
Batch Size 1 Kg

| Ingredients in W.D Order | %/Mix | Function |
|---|---|---|
| DE IONISED WATER | NA | Bulking Agent |
| MAGNESIUM LACTATE | 17.000 | Active |
| CAPRYLIC.CAPRIC/TRIGLYCERIDES | NA | Emulsifier |
| (EDIBLE) | | |
| CETEARYL ALCOHOL | NA | Emulsifier |
| GLYCERYL MONOSTEARATE-NON GMO | | |
| PALM OIL-POWDER | NA | Emulsifier |
| ISOPROPYL MYRISTATE (IPM) | NA | Emollient |
| CETEARETH-20 | NA | Emulsifier |
| CETYL ALCOHOL | NA | Emulsifier |
| 2 PHENOXYETHANOL3(2- | NA | Preservative |
| ETHYLHEXYLOXY)PROPANE-1,2-DIOL | | |
| BLACK PEPPER | 0.500 | Active |
| TURMERIC | 0.500 | Active |
| IMIDAZOLIDINYL UREA | NA | Preservative |
| CAPSICUM | 0.010 | Active |

Characteristics
Colour: YELLOW CREAM
Odour: CHARACTERISTIC
Shelf Life: 2 Years Minimum

### Example 3

In a third example, the invention relates to a warming cream formulation comprising 7 weight % of magnesium L-lactate, 1.0 weight % of turmeric gl-1 ps liquid extract, 0.5 weight % of turmeric, and 1.0 weight % of capsicum oleoresin 6%. The rest of the composition is made up of the bulking agents, emollients, thickening agents, viscosity modifiers, emulsifiers, and preservatives.

SPECIFICATION - Formulation 3 Warming Cream
Product Name: MAGNESIUM LACTATE CREAM - Warming
Batch Size 100 ml

| Ingredients in W.D Order | %/Mix | Function |
|---|---|---|
| DE IONISED WATER | NA | Bulking Agent |
| MAGNESIUM LACTATE | 7.000 | Active |
| GLYCERYL MONOSTEARATE- | | |
| NON GMO | | |
| PALM OIL-POWDER | NA | Thickening agent |
| | | Consistency |
| CETEARYL ALCOHOL | NA | Factor |
| CAPRYLIC.CAPRIC/TRIGLYCERIDES | NA | Emollient |
| (EDIBLE) MCT | | |
| ISOPROPYL MYRISTATE (IPM) | NA | Emollient |
| CETEARETH-20 | NA | Emulsifier |
| CETYL ALCOHOL | NA | Viscosity Modifier |
| 2 PHENOXYETHANOL3(2- | NA | Preservative |
| ETHYLHEXYLOXY)PROPANE-1,2- | | |
| DIOL | | |
| CAPSICUM OLEORESIN 6% | 1.000 | Active |
| TURMERIC GL-1 PS LIQUID | 1.000 | Active |
| EXTRACT | | |

### Example 4

Patient 1 (male) suffered a fall injury to elbow and shoulder. The skin was not damaged. The cream of Example 1 was applied twice daily both to elbow and shoulder. The elbow was completely pain-free and without swelling within 4 days.

### Example 5

Patient 2 (female) suffered a fall injury to her knee. The cream of Example 1 was applied, and all swelling and pain subsided.

### Example 6

Patient 3 (male) treated ongoing pain and stiffness caused by my total hip replacement scar using the cream of Example 1. Reduction of pain was experienced.

### Example 7

Patient 4 (female) suffered from neuroma (inflamed nerve between toes three and four) which troubled her repeatedly over a number of years. The cream of Example 1 was applied on a daily basis, and the symptoms ameliorated.

### Example 8

Patient 5 (male) suffered from chronic low back pain. Daily application of the cream of Example 1 resulted in relief of the pain.

### Example 9

A cohort of two patients (one male, on female) suffering from muscular pain caused by exercise applied the cream of Example 1 twice daily. The pain experienced was reduced within 7 days.

### Example 10

In a third example, the invention relates to a Magnesium Lactate cream comprising 8.0 weight % of magnesium L-lactate. The rest of the composition is made up of the bulking agent, emollient, emulsifiers, and preservatives.

SPECIFICATION - Formulation 4
Product Name: MAGNESIUM LACTATE CREAM
Batch Size 1 kg

| Ingredients in W.D Order | %/Mix | Function |
|---|---|---|
| DE IONISED WATER | NA | Bulking Agent |
| MAGNESIUM LACTATE | 8.000 | Active |
| GLYCERYL MONOSTEARATE-NON GMO | | |
| PALM OIL-POWDER | NA | Emulsifier |
| CETEARYL ALCOHOL | NA | Emulsifier |
| CAPRYLIC.CAPRIC/TRIGLYCERIDES | NA | Emulsifier |
| (EDIBLE) MCT | | |
| ISOPROPYL MYRISTATE (IPM) | NA | Emollient |
| CETEARETH-20 | NA | Emulsifier |
| CETYL ALCOHOL | NA | Emulsifier |
| 2 PHENOXYETHANOL3(2- | NA | Preservative |
| ETHYLHEXYLOXY)PROPANE-1,2-DIOL | | |
| IMIDAZOLIDINYL UREA | NA | Preservative |

### Example 11

Patient 8 (healthy male, mid 70s) who developed a stress fracture of the Right 5th metatarsal, applied the cream of example 10 twice daily and the pain was completely resolved after 5 weeks with healing of the stress fracture.

### Example 12

Patient 9 (female) who has been battling chronic “shin splints” for several years in the left leg and has been in pain for a long time applied the cream of example 10 twice a day and the pain experienced was significantly reduced within 3 weeks.

### Example 13

Patient 10 (male, age 75) fell off e-bike and landed or right elbow with extensive bruising and sprain. Application of the cream of example 10 twice daily resulted in almost immediate resolution of pain (24 hours) with improvement in swelling and function within a few days—healing occurred without pain within half the expected time.

### Example 14

Patient 11 (male, age 75) suffered from chronic low back pain rendering him unable to bend down, and neck pain with limited rotation and pain on turning to right. The application of the cream of example 10 led to improved function with near normal rotation of neck and minimal pain and he is now able to bend and lift freely. First noticeable improvements were reported within 24-48 hours.

### Example 15

Patient 12 (female, age 59) suffered from post-surgical chronic pain in both knees. The application of the cream of example 10 led to improved function with noticeable improvements after 24-28 hours.

### Example 16

Patient 13 (female, age 30) sprained an ankle and applied the cream of example 10 twice daily which resulted in markedly reduced pain and the ankle was healed within 2 days.

### Example 17

The cream of example 10 was investigated with a cohort of patients with recalcitrant neuropathic post traumatic pain. The early responses have been encouraging with positive feedback from the patients. One particular example:
A 74-year-old female patient had chronic lateral ankle pain, in particular nocturnal, following a sprain. Overnight application of the cream relieved the pain.

### Example 18

Patient 14 (female, early 60s) had a long history of recurring debilitating pain in neck and shoulders, usually triggered by a bad sleeping position. The cream of example 10 was applied two to three times over two days and the debilitating pain was reported to have been rapidly subsided and by day three it was reported to have completely disappeared.

Various modifications will be apparent to those skilled in the art. For example, any known emulsifiers, emollients, moisturisers, preservatives, pH adjusters, or the like can be included in the preparation of the pain relief formulation comprising magnesium L-lactate. Another example may be that whilst the above composition is suitable for use in human, it can be also used in treating animals.

1-12. (canceled)

13. A method to treat pain, comprising topically administering a composition comprising an effective amount of magnesium L-lactate or a solvate thereof and one or more pharmaceutically acceptable excipients.

14. The method of claim 13, wherein the composition comprises magnesium L-lactate at a concentration of 5.0 to 25.0 weight percent.

15. The method of claim 13, wherein the composition comprises vitamin D.

16. The method of claim 15, wherein the composition comprises the vitamin D at an amount up to 40,000 International Units per milliliter.

17. The method of claim 13, wherein the composition is a topical analgesic that is provided in the form of a gel, ointment, lotion, emulsion, cream, foam, mousse, liquid, spray, suspension, dispersion, or aerosol.

18. The method of claim 13, wherein the composition comprises medium-chain triglycerides.

19. The method of claim 18, wherein the medium-chain triglycerides are caprylic/capric triglycerides.

20. The method of claim 13, wherein the composition comprises solid aliphatic alcohols.

21. The method of claim 20, wherein the solid aliphatic alcohols are selected from stearyl alcohols, cetyl alcohols, and mixtures thereof.

22. The method of claim 13, wherein the composition comprises one or both of mono-glycerides of long chain fatty acids and di-glycerides of long chain fatty acids.

23. The method of claim 22, wherein the composition comprises glyceryl monostearate.

24. The method of claim 13, wherein the composition comprises one or more polyoxyethylene alkyl ethers.

25. The method of claim 24, wherein the one or more polyoxyethylene alkyl ethers comprise polyethylene glycol ether of cetearyl alcohol (Ceteareth-20).

26. The method of claim 13, wherein the composition comprises one or more preservatives.

27. The method of claim 26, wherein the one or more preservatives comprise 3-(2-ethylhexyloxy)propane-1,2-diol, imidazolidinyl urea, and mixtures thereof.

28. The method of claim 13, wherein the composition comprises either (1) one or more cooling additives or (2) one or more warming additives.

29. The method of claim 28, wherein the composition comprises one or both of boswellia extract and eucalyptus essential oil.

30. The method of claim 28, wherein the composition comprises one, two, or each of black pepper essential oil, turmeric, and capsicum.
